(19) United States
(12) Patent Application Publication
(10) Pub. No.: US 2014/0274871 A1
(43) Pub. Date: Sep. 18, 2014

(54) LOW ESTER PECTIN AIR FRESHENER GELS AND METHODS FOR MAKING THE SAME

(71) Applicant: CP KELCO APS, Lille Skensved (DK)

(72) Inventors: Jens Eskil Trudsoe, Roskilde (DK); Hanne Thulstrup Jensen, Klippinge (DK)

(73) Assignee: CP KELCO APS, Lille Skensved (DK)

(21) Appl. No.: 14/189,222

(22) Filed: Feb. 25, 2014

Related U.S. Application Data
(60) Provisional application No. 61/792,735, filed on Mar. 15, 2013.
(60) Provisional application No. 61/802,985, filed on Mar. 18, 2013.

(51) Int. Cl. A61L 9/04

(52) U.S. Cl. CPC A61L 9/048
USPC 512/4

(57) ABSTRACT

Air freshening gel compositions include a low ester pectin in an amount from about 0.5 to about 2.5 percent by weight, a substantially insoluble calcium salt in an amount from about 0.1 to about 0.50 percent by weight, water, and fragrance. Methods of making an air freshening gel composition include dissolving a low ester pectin in water, and combining a calcium salt with the pectin solution to form a gel solution containing from about 0.5 to about 2.5 percent by weight of the low ester pectin and from about 0.1 to about 0.50 percent by weight of the calcium salt.

### CROSS-REFERENCE TO RELATED APPLICATIONS

This application claims priority to U.S. Provisional Patent Application Ser. No. 61/792,735, filed on Mar. 15, 2013, and Ser. No. 61/802,985, filed on Mar. 18, 2013, which are incorporated by reference herein.

### TECHNICAL FIELD

The invention relates to the field of gels and more particularly relates to low ester pectin air freshener gels and methods for making the same.

### BACKGROUND

Gels are used in many applications including cleaning products, such as for surfaces, fabrics, and the like, and air treatment materials for continuous release of air treatment components such as perfumes, disinfectants, bactericides, insecticides, and the like.

Certain gels may be made without the use of heating and cooling. In general, the known techniques for making cold prepared (i.e., prepared at room temperature) air freshener gels rely on complex chemistry using components that are toxic or hazardous. Therefore, a need exists for more environmentally friendly and non-toxic air freshener gels and methods of making cold prepared air freshener gels.

### SUMMARY

In one aspect, an air freshening gel composition is provided. The gel composition includes a low ester pectin in an amount from about 0.5 to about 2.5 percent by weight of the gel composition, a substantially insoluble calcium salt in an amount from about 0.1 to about 0.50 percent by weight of the gel composition, water, and fragrance.

In another aspect, methods of making air freshening gel compositions are provided. In one embodiment, a method includes: (i) dissolving a low ester pectin in demineralized water to form a pectin solution, (ii) suspending a substantially insoluble calcium salt in demineralized water to form a salt suspension, (iii) combining the pectin solution and the salt suspension to form a gel solution, and (iv) allowing the gel solution to form a gel composition, wherein the low ester pectin is present in the gel solution in an amount from about 0.5 to about 2.5 percent by weight of the gel solution, and the calcium salt is present in the gel solution an amount from about 0.1 to about 0.50 percent by weight of the gel solution. In one embodiment, a method includes: (i) dissolving a low ester pectin in water to form a pectin solution, (ii) adding a calcium salt to the pectin solution to form a gel solution, and (iii) allowing the gel solution to form a gel composition, wherein the low ester pectin is present in the gel solution in an amount from about 0.5 to about 2.5 percent by weight of the gel solution, and the calcium salt is present in the gel solution an amount from about 0.1 to about 0.50 percent by weight of the gel solution.

### BRIEF DESCRIPTION OF THE DRAWINGS

FIG. 1 is a graph showing gel strength over time for 1% GENU® pectin CS 30 gels having varying concentrations of calcium sulfate.

FIG. 2 is a graph showing gel strength over time for 2% GENU® pectin CS 30 gels having varying concentrations of calcium sulfate.

FIG. 3 is a graph showing gel strength over time for 2% GENU® pectin CS 30 gels having varying concentrations of calcium hydrogen phosphate.

FIG. 4 is a graph showing gel strength over time for 2% GENU® pectin CS 30 gels having varying concentrations of calcium citrate.

FIG. 5 is a graph showing gel strength over time for 2% GENU® pectin CS 30 gels having varying concentrations of tri-calcium phosphate.

FIG. 6 is a graph showing the gelling time for gel solutions containing varying amounts of sodium hexametaphosphate.

FIG. 7 is a graph showing the gelling time for gel solutions having varying pH.

FIG. 8 is a graph showing the gelling time for gel solutions containing 0.005 weight percent sodium hexametaphosphate at varying pH.

FIG. 9 is a graph showing the gel strength of gel compositions that were stirred for varying amounts of time.

### DETAILED DESCRIPTION

The present invention addresses the above-described need by providing air freshener gels and methods of making air freshener gels using low ester pectins. Several embodiments of this invention are described below including an air freshener gel, although the embodiments of this invention may also be used for other applications such as food products. Parameters of different steps, components, and products of the embodiments are described separately, but may be combined consistently with this description and claims to enable still other embodiments as will be understood by those skilled in the art. Through the specification, examples, and claims, unless otherwise indicated, percents are percents by weight.

As used herein the term “low ester pectin” refers to pectins having less than 50% ester groups (e.g., pectins having a degree of esterification or “DE” value of below 50). Low ester pectins are weak acids that can be soluble in cold (i.e., room temperature) demineralized water, and can be reactive with certain ions to form gels without the need of sugar or other soluble solids. As used herein the term “room temperature” refers to a temperature of about 20° C. to about 25° C. Embodiments of the gel compositions and methods for making embodiments of the gel compositions are described in detail.

Gel Compositions

In certain embodiments, the air freshening gel composition includes a low ester pectin, a substantially insoluble calcium salt, water, and fragrance. The term “substantially insoluble” means that the calcium salt should be sparingly soluble or have limited solubility in water. For example, the air freshening gel composition may include a low ester pectin in an amount from about 0.5 to about 2.5 percent by weight of the gel composition, and a substantially insoluble calcium salt in an amount from about 0.1 to about 0.50 percent by weight of the gel composition. In one embodiment, the low ester pectin is present in an amount from about 1.0 to about 2.0 percent by weight of the gel composition, and the calcium salt is present in an amount from about 0.1 to about 0.4 percent by weight of the gel composition. In one embodiment, the low ester pectin is present in an amount of about 2.0 percent by weight of the gel composition, and the calcium salt is present in an amount of about 0.15 percent by weight of the gel composition.

In certain embodiments, the low ester pectin has a degree of esterification (“DE”) of below 40. For example, the low ester pectin may have a DE of about 25 to about 40, about 25 to about 35, or about 30 to about 35. For example the low ester pectin may be GENU® pectin CS 30, which is commercially available from CP Kelco U.S., Inc. (Atlanta, Ga.).

In certain embodiments, the substantially insoluble calcium salt may be calcium citrate, tri-calcium phosphate, calcium sulfate, calcium hydrogen phosphate, or a combination thereof.

In certain embodiments, the air freshening gel composition further includes a fragrance substance. For example, the fragrance substance may be grape fruit oil, lemon oil, or a combination thereof. In one embodiment, the fragrance substance is present in an amount from about 4.0 to about 6.0 percent by weight of the gel composition. In one embodiment, the fragrance substance is present in an amount from about 4.5 to about 5.5 percent by weight of the gel composition.

In certain embodiments, the gel composition has a gel strength from about 5 grams to about 250 grams. For example, the gel composition may have a gel strength from about 30 grams to about 230 grams, or from about 5 grams to about 100 grams. Gel strength is measured using a TA.XT Texture Analyzer from Stable Micro Systems, equipped with a one inch plunger. Gel strength is defined as the load required to deform the gel by 2 mm when the plunger is driven into the gel at a speed of 1 mm per second.

In certain embodiments, the gel composition has a pH from about 3.0 to about 5.5. For example, the gel composition may have a pH from about 3.5 to about 5.5.

Methods of Making the Gel Compositions

In certain embodiments, a method of making an air freshening gel composition includes: (i) dissolving a low ester pectin in water to form a pectin solution, (ii) adding a calcium salt to the pectin solution to form a gel solution, and (iii) allowing the gel solution to form a gel composition. In certain embodiments, the method includes suspending the calcium salt in water to form a salt suspension and combining the pectin solution and the salt suspension to form a gel solution. In certain embodiments, the water is demineralized.

In one embodiment, the low ester pectin is present in the gel solution in an amount from about 0.5 to about 2.5 percent by weight of the gel solution, and the calcium salt is present in the gel solution an amount from about 0.1 to about 0.50 percent by weight of the gel solution. In one embodiment, the low ester pectin is present in an amount from about 1.0 to about 2.0 percent by weight of the gel solution, and the calcium salt is present in an amount from about 0.1 to about 0.4 percent by weight of the gel solution. In one embodiment, the low ester pectin is present in an amount of about 2.0 percent by weight of the gel solution, and the calcium salt is present in an amount of about 0.15 percent by weight of the gel composition.

In certain embodiments, the methods also include dispersing a fragrance substance with the low ester pectin and calcium salt in the demineralized water to form the gel solution. For example, the step of suspending the calcium salt in demineralized water may include dispersing a fragrance substance in the demineralized water with the calcium salt to form the salt suspension. Alternatively, the step of dissolving a low ester pectin in demineralized water to form a pectin solution may include dispersing a fragrance substance in the demineralized water with the pectin to form the pectin solution. In one embodiment, the fragrance substance is present in the gel solution in an amount from about 4.0 to about 6.0 percent by weight of the gel solution. In one embodiment, the fragrance substance is present in the gel solution an amount from about 4.5 to about 5.5 percent by weight of the gel solution.

In certain embodiments, the demineralized water is at room temperature. That is, the water, the low ester pectin and/or calcium salt are dissolved and/or suspended at a temperature of about 20 to about 25° C.

In certain embodiments, the dissolving steps include stirring the solutions. For example, the solutions may be stirred by a magnetic stirrer.

In certain embodiments, the gel composition is formed from the gel solution in from about 2 to about 5 minutes. Advantageously, the time for gelling may be controlled within the range of about 2 to about 5 minutes, which allows for stabilization of an oil or a fragrance in the gel composition, so that the oil or fragrance does not leak to the surface of the gel composition.

In certain embodiments, the methods also include adding a sugar beet pectin solution to form the gel solution. For example, the sugar beet pectin solution may be added to the pectin/fragrance solution prior to combining the pectin/fragrance solution and the salt suspension. In one embodiment, the sugar beet pectin solution contains sugar beet pectin in an amount from about 1.0 to about 3.0 percent by weight of the sugar beet pectin solution. In one embodiment, the sugar beet pectin solution is present in the gel solution in an amount from about 0.25 to about 1.25 percent by weight of the gel solution. For example, a sugar beet pectin solution may be used in compositions where improved emulsification is desired. For example, a small concentration of sugar beet pectin may be added to the gel solution in solution form, because sugar beet pectin is not soluble in cold demineralized water.

It was found that a gel solution utilizing low ester pectin, demineralized water, and a substantially insoluble calcium salt, enabled one to make non-toxic air freshener gels without the use of heating and cooling. Furthermore, the use of a substantially insoluble calcium salt advantageously allows the weak acidity of low ester pectin to dissolve the calcium salt slowly enough to avoid breakage of the gel during mixing of the low ester pectin solution with a suspension of the substantially insoluble calcium salt. For example, calcium hydrogen phosphate, calcium sulfate, calcium citrate, and tri-calcium phosphate are sufficiently slowly soluble to avoid pre-gelation during mixing of the low ester pectin solution and the suspension of the calcium salt.

Gel compositions made by embodiments of the methods described herein have the advantage of evaporating completely over time. This allows consumers to easily determine the lifetime of the air freshener gel or product and when a new product is required.

As compared to air freshener gels made from high ester pectins, gel compositions made from low ester pectins do not require both sugar/soluble solids and acid to form a gel. Because high ester pectin based gels contain about 50 percent soluble solids, the gel can only shrink or evaporate by 50 percent over its lifetime. Thus, the present gels provide a distinct advantage over gels made using high ester pectins.

In certain embodiments, it is desirable to slow down the gelation time of these compositions, for example to allow for additional processing time. In one embodiment, the gelation time of the composition may be slowed by adding a calcium stabilizing-sequestrant, such as sodium hexametaphosphate or sodium pyrophosphate, among others, to the gel solution. In one embodiment, the gelation time of the composition may be slowed by adding a pH adjustor, for example a strong base, such as sodium hydroxide, among others, to increase the pH of the composition and thereby reduce the solubility of the calcium salt. For example, the sequestrant and/or pH adjustor may be combined with the pectin and/or calcium salt via an in-line mixing process, as part of the pectin solution, as part of the salt suspension, or directly in the gel solution.

In one embodiment, a method includes adding a calcium-stabilizing sequestrant and/or a pH adjustor to the gel solution to increase a gel formation time of the gel composition. For example, adding the calcium-stabilizing sequestrant and/or the pH adjustor to the gel solution may include adding the calcium-stabilizing sequestrant and/or the pH adjustor to the pectin solution. In one embodiment, a method includes adding sodium hexametaphosphate to the pectin solution, wherein the sodium hexametaphosphate is present in the gel solution in an amount from about 0.001 to about 0.01 percent by weight of the gel solution. In one embodiment, a method includes adding sodium hydroxide to the pectin solution, wherein the sodium hydroxide is present in the gel solution in an amount effective to bring a pH of the gel solution within a range from about 4.0 to about 4.7.

For example, a sequestrant such as sodium hexametaphosphate may be provided in the gel solution in an amount from about 0.002 to about 0.005 weight percent. For example, a pH adjustor may be used to adjust the pH of the gel solution to a pH of from about 3.5 to about 5.0. In certain embodiments, a sequestrant and a pH adjustor are used in combination to increase the gelation time of the gel composition.

### EXAMPLES

The present invention is further illustrated by the following examples, which are not to be construed in any way as imparting limitations upon the scope thereof. On the contrary, it is to be clearly understood that resort may be had to various other embodiments, modifications, and equivalents thereof which, after reading the description therein, may suggest themselves to those skilled in the art without departing from the scope of the appended claims.

Gels were made in accordance with the following composition parameters: 100 ml demineralized water, 1-2 g low ester pectin, 0.05-0.40 g calcium salt, 5 ml fragrance/oil, and 0.5-1 ml 2% sugar beet pectin solution. Gels were made in accordance with the following method steps: (i) pectin was dispersed in 90 ml cold (room temperature) demineralized water while stirring on magnetic stirrer, stirring was continued until pectin was dissolved, (ii) calcium salt and oil/fragrance was dispersed in 10 ml cold demineralized water while stirring on magnetic stirrer, (iii) optionally 0.5-2 ml 2% sugar beet solution was added to the dispersion of calcium salt and oil/fragrance or to the pectin solution (the sugar beet pectin solution was heated to 70° C. and cooled to room temperature prior to use), (iv) the pectin solution was poured into the dispersion of calcium salt and oil/fragrance while stirring vigorously, and (v) the mixture was left standing under quiescent conditions. The time until visually-observable gelation was recorded, the gel strength was measured over time on a TA.XT Texture Analyzer from Stable Micro Systems, equipped with a one inch plunger, which was driven into the gel at a speed of 1 mm per second until the gel was deformed by 2 mm, and the pH of gels was measured.

### Example 1

In the first set of experiments, anhydrous calcium sulfate was used at various concentrations, and GENU®pectin CS 30, which has a DE value of 30, was used in 1% concentration. The gel strength was measured and visual observations of the gel appearance were made, over a period of about three days. The results are shown in Tables 1 and 2.

TABLE 1
Gel strength (GS) over time with 1% GENU ® pectin
CS 30 and different concentrations of calcium sulfate.

| | | | | Mixed | | | | | | |
|---|---|---|---|---|---|---|---|---|---|---|
| | | CaSO₄ | | Time | | | | | Gel-time | |
| Sample | Pectin % | g | pH | (Day 1) | GS1 | GS2 | GS3 | GS4 | min | Comments |
| 1 | 1 | 0.05 | 3.73 | 08:50 | 4 | 4 | | | 8 | Weak gel |
| 2 | 1 | 0.10 | 3.62 | 08:51 | 18 | 21 | 24 | 24 | 1 | |
| 3 | 1 | 0.20 | 3.47 | 09:35 | 87 | 111 | 105 | 71 | <1 | Pregelled |
| 4 | 1 | 0.40 | 3.42 | 09:36 | 112 | 121 | 70 | 59 | <1 | Pregelled |
| 5 | 1 | 0.15 | 3.56 | 10:10 | 45 | 69 | 76 | 78 | >1 | Strong gel |

TABLE 2
Comments over time with 1% GENU ® pectin CS 30
and different concentrations of calcium sulfate.

| Sample | Day | GS | Time | Day | GS | Time | Comment |
|---|---|---|---|---|---|---|---|
| 1 | 2 | 1 | 14:10 | 3 | 2 | 12:52 | Weak |
| 2 | 1 | 1 | 09:50 | 1 | 2 | 14:55 | OK |
| 3 | 1 | 1 | 10:05 | 1 | 2 | 14:57 | OK |
| 4 | 1 | 1 | 10:06 | 1 | 2 | 14:58 | Syneresis |
| 5 | 1 | 1 | 10:35 | 1 | 2 | 15:00 | OK |

| Sample | Day | GS | Time | Comment | Day | GS | Time | Comment |
|---|---|---|---|---|---|---|---|---|
| 1 | | 3 | | | | 4 | | |
| 2 | 2 | 3 | 14:15 | OK | 3 | 4 | 12:55 | OK - self |
| | | | | | | | | supporting |
| 3 | 2 | 3 | 14:16 | Syneresis | 3 | 4 | 12:57 | Syneresis - |
| | | | | | | | | Self |
| | | | | | | | | supporting |
| 4 | 2 | 3 | 14:18 | Syneresis | 3 | 4 | 13:00 | Syneresis - |
| | | | | | | | | Self |
| | | | | | | | | supporting |
| 5 | 2 | 3 | 14:20 | OK | 3 | 4 | 13:02 | OK - self |
| | | | | | | | | supporting |

### Example 2

In the next set of experiments, the concentration of GENU® pectin CS 30 was increased to 2%, and gel strength was measured over time. The results are shown in Tables 3 and 4.

TABLE 3
Gel strength over time with 2% GENU ® pectin CS
30 and different concentrations of calcium sulfate.

| | | | | Mixed | | | | | | |
|---|---|---|---|---|---|---|---|---|---|---|
| | | CaSO₄ | | Time | | | | | Gel-time | |
| Sample | Pectin % | g | pH | (Day 1) | GS1 | GS2 | GS3 | GS4 | min | Comments |
| 1 | 2 | 0.10 | 3.61 | 10:25 | 10 | 12 | 15 | 16 | <1 min | Weak, dry, |
| | | | | | | | | | | bubbles |
| 2 | 2 | 0.15 | 3.53 | 10:25 | 84 | 111 | 135 | 141 | <1 min | Strong, dry, |
| | | | | | | | | | | bubbles, |
| | | | | | | | | | | does not |
| | | | | | | | | | | melt at |
| | | | | | | | | | | 90° C. |
| 3 | 2 | 0.20 | 3.48 | 10:30 | 85 | 111 | 141 | | pregel | Syneresis |
| 4 | 2 | 0.30 | 3.40 | 10:30 | 330 | 384 | 291 | | pregel | Syneresis |

TABLE 4
Comments over time with 2% GENU ® pectin CS 30
and different concentrations of calcium sulfate

| Sample | Day | GS | Time | Day | GS | Time | Comment |
|---|---|---|---|---|---|---|---|
| 1 | 1 | 1 | 11:00 | 1 | 2 | 15:00 | Syneresis |
| 2 | 1 | 1 | 11:00 | 1 | 2 | 15:00 | Syneresis |
| 3 | 1 | 1 | 11:55 | 1 | 2 | 15:20 | OK |
| 4 | 1 | 1 | 11:55 | 1 | 2 | 15:20 | OK |

| Sample | Day | GS | Time | Comment | Day | GS | Time | Comment |
|---|---|---|---|---|---|---|---|---|
| 1 | 2 | 3 | 11:00 | Syneresis | | | | |
| 2 | 2 | 3 | 11:00 | Syneresis | | | | |
| 3 | 2 | 3 | 13:15 | OK | 3 | 4 | 9:45 | OK |
| 4 | 2 | 3 | 13:15 | OK | 3 | 4 | 9:45 | OK |

### Example 3

In the next experiments, calcium hydrogen phosphate dihydrate was used. The results of the gel strength and visual observation tests are shown in Tables 5 and 6.

TABLE 5
Gel strength over time with 2% GENU ® pectin CS 30 and
different concentrations of calcium hydrogen phosphate.

| | | | | Mixed | | | | | | |
|---|---|---|---|---|---|---|---|---|---|---|
| | | CaHPO₄ | | Time | | | | | Gel-time | |
| Sample | Pectin % | g | pH | (Day 1) | GS1 | GS2 | GS3 | GS4 | min | Comments |
| 1 | 2 | 0.15 | 3.91 | 9:40 | 20 | 30 | 35 | 36 | <2 min | Dry, bubbles |
| 2 | 2 | 0.20 | 4.07 | 9:40 | 55 | 78 | 88 | 86 | <1 min | Dry, bubbles |

TABLE 6
Comments over time with 2% GENU ® pectin CS 30 and
different concentrations of calcium hydrogen phosphate.

| Sample | Day | GS | Time | Day | GS | Time | Comment |
|---|---|---|---|---|---|---|---|
| 1 | 1 | 1 | 10:10 | 1 | 2 | 14:35 | Dry |
| 2 | 1 | 1 | 10:10 | 1 | 2 | 14:35 | Dry |

| Sample | Day | GS | Time | Comment | Day | GS | Time | Comment |
|---|---|---|---|---|---|---|---|---|
| 1 | 2 | 3 | 14:15 | Dry | 3 | 4 | 15:00 | Sags |
| 2 | 2 | 3 | 14:15 | Dry | 3 | 4 | 15:00 | Self |
| | | | | | | | | supporting |

### Example 4

In the next experiments, calcium citrate tetra hydrate was used. The results of the gel strength and visual observation tests are shown in Tables 7 and 8.

TABLE 7
Gel strength over time with 2% GENU ® pectin CS
30 and different concentrations of calcium citrate.

| | | | | Mixed | | | | | | |
|---|---|---|---|---|---|---|---|---|---|---|
| | | Ca-citrate | | Time | | | | | Gel- time | |
| Sample | Pectin % | g | pH | (Day 1) | GS1 | GS2 | GS3 | GS4 | min | Comments |
| 1 | 2 | 0.15 | 4.06 | 10:25 | Weak | 10 | 12 | 13 | <10 minutes | Some wet, |
| | | | | | | | | | | no bubbles |
| 2 | 2 | 0.20 | 4.24 | 10:25 | 24 | 55 | 67 | 67 | <5 minutes | Dry, no |
| | | | | | | | | | | bubbles |

TABLE 8
Comments over time with 2% GENU ® pectin CS 30
and different concentrations of calcium citrate.

| Sample | Day | GS | Time | Day | GS | Time | Comment |
|---|---|---|---|---|---|---|---|
| 1 | 1 | 1 | 11:00 | 1 | 2 | 15:00 | Dry |
| 2 | 1 | 1 | 11:00 | 1 | 2 | 15:00 | Dry |

| Sample | Day | GS | Time | Comment | Day | GS | Time | Comment |
|---|---|---|---|---|---|---|---|---|
| 1 | 2 | 3 | 14:30 | Some wet | 3 | 4 | 13:00 | Sags - |
| | | | | | | | | some wet |
| 2 | 2 | 3 | 14:30 | Dry | 3 | 4 | 13:00 | Self |
| | | | | | | | | supporting - |
| | | | | | | | | Dry |

### Example 5

In the next experiments, tri-calcium phosphate was used. The results of the gel strength and visual observation tests are shown in Tables 9 and 10.

TABLE 9
Gel strength over time with 2% GENU ® pectin CS 30
and different concentrations of tri-calcium phosphate.

| | | | | Mixed | | | | | Gel- | |
|---|---|---|---|---|---|---|---|---|---|---|
| | | | | Time | | | | | time | |
| Sample | Pectin % | Ca₃(PO₄)₂ | pH | (Day 1) | GS1 | GS2 | GS3 | GS4 | min | Comments |
| 1 | 2 | 0.05 | 4.33 | 09:35 | 4 | 5 | 5 | 6 | 5 | No bubles; |
| | | | | | | | | | | dry; gummy |
| 2 | 2 | 0.1 | 4.46 | 09:35 | 22 | 29 | 33 | 34 | 3 | No bubles; |
| | | | | | | | | | | dry; gummy |
| 3 | 2 | 0.15 | 5.17 | 09:35 | 55 | 86 | 97 | 101 | 2 | No bubles; |
| | | | | | | | | | | dry; gummy |

TABLE 10
Comments over time with 2% GENU ® pectin CS 30 and
different concentrations of tri-calcium phosphate.

| Sample | Day | GS | Time | Day | GS | Time | Comment |
|---|---|---|---|---|---|---|---|
| 1 | 1 | 1 | 10:05 | 1 | 2 | 14:50 | Weak |
| 2 | 1 | 1 | 10:05 | 1 | 2 | 14:50 | Firm |
| 3 | 1 | 1 | 10:05 | 1 | 2 | 14:50 | Firm |

| Sample | Day | GS | Time | Comment | Day | GS | Time | Comment |
|---|---|---|---|---|---|---|---|---|
| 1 | 2 | 3 | 13:35 | Weak | 3 | 4 | 13:35 | Weak |
| 2 | 2 | 3 | 13:35 | Firm | 3 | 4 | 13:35 | Self |
| | | | | | | | | supporting |
| 3 | 2 | 3 | 13:35 | Firm | 3 | 4 | 13:35 | Self |
| | | | | | | | | supporting |

### Example 6

A single test was done using 2 g GENU® pectin 12 CG (supplied by CP Kelco U.S., Inc.) having a DE of about 35 in 90 ml demineralized water and 0.15 g tri-calcium phosphate in 10 ml demineralized water. This pectin product was not completely dissolved in the cold water, and after 1 hour, the gel was firm and dry, but the gel was clearly pre-gelled with bubbles.

### Example 7

Next, the effect of sugar beet pectin was investigated. Sugar beet pectin is not soluble in cold demineralized water, so a 2% stock solution of sugar beet pectin was made up by dispersing 2 g sugar beet pectin (GENU® pectin Beta, available from CP Kelco U.S., Inc.) in cold demineralized water and heating the dispersion while stirring to 70° C. The solution was then cooled in 5° C. cooling bath to room temperature (about 20-about 25° C.). The stability of an emulsion was determined by (i) adding a specific volume of 2% sugar beet pectin (SBP) into a beaker, (ii) adding 5 ml grape fruit oil (fragrance substance), (iii) adding 90 ml demineralized water, (iv) emulsifying the mixture with a high speed mixer (Silent Crusher, 8000 rpm for 5 minutes), and (v) visually evaluating the stability of the emulsion over time. The results of this test are shown in Table 11.

TABLE 11
Evaluation of emulsions with sugar beet pectin.

| 2% SBP | After | |
|---|---|---|
| ml | Minutes | Evaluation |
| 0 | 0 | Not quite milky; oil drops on surface |
| | 5 | About 1 cm clear phase in bottom; oil drops on surface |
| | 10 | About 0.1 cm oil phase in top |
| 0.5 | 0 | Milky; fine emulsion; no oil drops on surface |
| | 5 | About 0.5 cm clear phase in bottom; no oil drops on |
| | | surface |
| | 10 | About 1 cm clear phase in bottom; no oil drops on |
| | | surface |
| | 15 | About 1.5 cm clear phase in bottom; no oil drops on |
| | | surface |
| | 20 | About 2 cm clear phase in bottom; no oil drops on |
| | | surface |
| 1 | 0 | Milky; fine emulsion; no oil drops on surface |
| | 5 | About 0.2 cm clear phase in bottom; no oil drops on |
| | | surface |
| | 10 | About 0.5 cm clear phase in bottom; no oil drops on |
| | | surface |
| | 15 | About 1 cm clear phase in bottom; no oil drops on |
| | | surface |
| | 20 | About 1.5 cm clear phase in bottom; no oil drops on |
| | | surface |
| 2 | 0 | Milky; fine emulsion; no oil drops on surface |
| | 5 | About 0.2 cm clear phase in bottom; no oil drops on |
| | | surface |
| | 10 | About 0.5 cm clear phase in bottom; no oil drops on |
| | | surface |
| | 15 | About 0.5 cm clear phase in bottom; no oil drops on |
| | | surface |
| | 20 | About 1 cm clear phase in bottom; no oil drops on |
| | | surface |

Based on these evaluations, two cold prepared gels were made containing a fragrance substance, one with sugar beet pectin and one without, according to the following parameters.

A cold prepared gel without sugar beet pectin was prepared with 2 g GENU® pectin CS 30, 0.15 g Tri calcium phosphate in 10 ml demineralized water, 90 ml demineralized water, and 5 ml grape fruit oil. The gel was prepared according to the following method: (i) GENU® pectin CS 30 was dissolved in 90 ml demineralized cold water at room temperature, (ii) grape fruit oil was added and the mixture was homogenized using Silent Crusher at 8000 rpm for 5 minutes, (iii) the homogenized mixture was poured into the dispersion of tri-calcium phosphate and mixed thoroughly on magnetic stirrer for a few seconds.

A cold prepared gel with sugar beet pectin was prepared with 2 g GENU® pectin CS 30, 0.15 g tri calcium phosphate in 10 ml demineralized water, 90 ml demineralized water, 5 ml grape fruit oil, and 1 ml 2% sugar beet pectin solution. The gel was prepared according to the following method: (i) GENU® pectin CS 30 30 was dissolved in 90 ml demineralized cold water at room temperature, (ii) sugar beet pectin solution was added, (iii) grape fruit oil was added and the mixture was homogenized using Silent Crusher at 8000 rpm for 5 minutes, and (iv) the homogenized mixture was poured into the dispersion of tri-calcium phosphate and mixed thoroughly on magnetic stirrer for a few seconds.

These preparations were repeated, but this time the oil was mixed with pectin solution(s) on magnetic stirrer for 5 minutes instead of using the Silent Crusher. The results of these tests are shown in Table 12.

TABLE 12
Effect of sugar beet pectin and emulsification of grape fruit oil.

| Sample | Emulsification | Evaluation |
|---|---|---|
| No sugar beet | Silent Crusher | Dry; no oil drops on |
| pectin | | surface |
| | Magnetic | Few oil drops on surface |
| | stirrer | |
| Sugar beet pectin | Silent Crusher | Dry; no oil drops on |
| | | surface |
| | Magnetic | Dry; no oil drops on |
| | stirrer | surface |

Subjectively, there was no difference in fragrance intensity between these gels.

The experiments using magnetic stirrer were done with Zesty fragrance instead of grape fruit oil. The results of these tests are shown in Table 13.

TABLE 13
Effect of sugar beet pectin on emulsification of Zesty fragrance.

| Sample | Emulsification | Evaluation |
|---|---|---|
| No sugar beet | Magnetic | Dry; no oil drops on |
| pectin | stirrer | surface |
| Sugar beet pectin | Magnetic | Dry; no oil drops on |
| | stirrer | surface |

Subjectively, there was no difference in fragrance intensity between these gels.

### Example 8

Next, it was attempted to slow down the time of gelation of the gel solution, to allow for additional processing time. First, a calcium sequestrant, sodium hexametaphosphate was used according to the composition shown in Table 14.

TABLE 14
Gel Composition Ingredients for Slowed Gelation Composition.

| Ingredients | % |
|---|---|
| De-ionized water | To 100 |
| GENU ® pectin CS 30 | 2.0 |
| Paraben | 0.08 |
| Glycerol | 10.0 |
| Fragrance | optional |
| Tri-calcium phosphate | 0.28 |
| De-ionized water (for tri-calcium phosphate) | 10.0 |
| Hexametaphosphate | 0-0.007 |

The gel composition was made by the following process: (i) mix gum, glycerol, and fragrance; (ii) add water; (iii) mix for 30 minutes; (iv) add parabens; (v) add hexametaphosphate for delayed gelation; (vi) homogenize on high speed mixer (Silverson with large holes) for 2 minutes; (vii) add slurry of water and tri-calcium phosphate to the solution and mix for 12 seconds; and (viii) pour into container. It was observed that addition of the sodium hexametaphosphate did not result in a change in the pH of the gel solution.

Next, the effect of pH on gelling time was tested. The above formulation and process used to test hexametaphosphate was used, except sodium hydroxide was used instead of the hexametaphosphate. The pH of the composition was adjusted to 3.80, 4.04, and 4.40.

Next, the combined effect of pH and hexametaphosphate on gelling time was tested. The concentration of hexametaphosphate was 0.005%.

### Example 9

The effect of stirring on the gel strength of gel compositions including 0.0025 weight percent of sodium hexametaphosphate was tested. Identical compositions were stirred for varying times before depositing for gel strength testing with a ½ inch plunger for deformation of 4 mm. It was determined that these gels could be stirred for up to about 30 minutes. The results of visual observations of the gel compositions are shown in Table 15.

TABLE 15
Effect of stirring on gel properties of compositions
with sodium hexametaphosphate.

| Stir Time | Observations |
|---|---|
| None | Smooth, dry gel |
| 15 minutes | Smooth, dry gel |
| 30 minutes | Slightly grainy, dry gel |
| 60 minutes | Grainy, dry gel |

### Experimental Results

Table 16 shows the gel strength over time using 1% GENU® pectin CS 30 and calcium sulfate.

TABLE 16
Gel strength over time with calcium sulfate.

| Sample | Day | Time | Time, min | GS |
|---|---|---|---|---|
| 1 | 1 | 08:50 | 0 | 0 |
| | 2 | 14:10 | 320 | 4 |
| | 3 | 12:52 | 1682 | 4 |
| 2 | 1 | 08:51 | 0 | 0 |
| | 1 | 09:50 | 59 | 18 |
| | 1 | 14:55 | 360 | 21 |
| | 2 | 14:15 | 1750 | 24 |
| | 3 | 12:55 | 3110 | 24 |
| 3 | 1 | 09:35 | 0 | 0 |
| | 1 | 10:05 | 30 | 87 |
| | 1 | 14:57 | 322 | 111 |
| | 2 | 14:16 | 1743 | 105 |
| | 3 | 12:57 | 3104 | 71 |
| 4 | 1 | 09:36 | 0 | 0 |
| | 1 | 10:06 | 30 | 112 |
| | 1 | 14:58 | 322 | 121 |
| | 2 | 14:18 | 1722 | 70 |
| | 3 | 13:00 | 3084 | 59 |
| 5 | 1 | 10:10 | 0 | 0 |
| | 1 | 10:35 | 25 | 45 |
| | 1 | 15:00 | 290 | 69 |
| | 2 | 14:20 | 1690 | 76 |
| | 3 | 13:02 | 3112 | 78 |

FIG. 1 shows how the gels firm up over time with 1% GENUS pectin CS 30 and different concentrations of calcium sulfate. The gel strength reaches its maximum after about 4 hours. As the amount of calcium sulfate exceeds about 0.15 g, the gel strength decreases over time. Without intending to be bound by a particular theory, it is believed that this is caused by substantial pre-gellation.

Table 17 shows the gel strength when using 2% GENU® pectin CS 30 with different amounts of calcium sulfate.

TABLE 17
Gel strength over time with 2% GENU ® pectin CS 30.

| Sample | Day | Time | Time, min | GS |
|---|---|---|---|---|
| 1 | 1 | 10:30 | 0 | 0 |
| | 1 | 11:00 | 30 | 85 |
| | 1 | 15:00 | 270 | 111 |
| | 2 | 11:00 | 1470 | 141 |
| 2 | 1 | 10:30 | 0 | 0 |
| | 1 | 11:00 | 30 | 330 |
| | 1 | 15:00 | 270 | 384 |
| | 2 | 11:00 | 1470 | 291 |
| 3 | 1 | 10:25 | 0 | 0 |
| | 1 | 11:55 | 90 | 10 |
| | 1 | 15:20 | 295 | 12 |
| | 2 | 13:15 | 1610 | 15 |
| | 3 | 9:45 | 2840 | 16 |
| 4 | 1 | 10:25 | 0 | 0 |
| | 1 | 11:55 | 90 | 84 |
| | 1 | 15:20 | 295 | 111 |
| | 2 | 13:15 | 1610 | 135 |
| | 3 | 9:45 | 2840 | 141 |

FIG. 2 shows how the gels firm up over time with 2% GENU® pectin CS 30 and different concentrations of calcium sulfate. Again the gel is almost complete after about 4 hours, but when the amount of calcium sulfate exceeds about 0.20 g, pre-gellation causes the gel strength to decrease over time.

Table 18 shows the data relating to gel strength over time when using calcium hydrogen phosphate.

TABLE 18
Gel strength over time with 2% GENU ® pectin CS 30
and calcium hydrogen phosphate.

| Sample | Day | Time | Time, min | GS |
|---|---|---|---|---|
| 1 | 1 | 9:40 | 0 | 0 |
| | 1 | 10:10 | 30 | 20 |
| | 1 | 14:35 | 295 | 30 |
| | 2 | 14:15 | 1715 | 35 |
| | 3 | 15:00 | 3200 | 36 |
| 2 | 1 | 9:40 | 0 | 0 |
| | 1 | 10:10 | 30 | 55 |
| | 1 | 14:35 | 295 | 78 |
| | 2 | 14:15 | 1715 | 88 |
| | 3 | 15:00 | 3200 | 86 |

FIG. 3 shows the gel strength over time with 2% GENU® pectin CS 30 and different concentrations of calcium hydrogen phosphate. The gel is basically completely formed after about 4 hours.

Table 19 shows the data relating to gel strength over time when using calcium citrate.

TABLE 19
Gel strength over time with calcium citrate.

| Sample | Day | Time | Time, min | GS |
|---|---|---|---|---|
| 1 | 1 | 10:25 | 0 | 0 |
| | 1 | 11:00 | 35 | 0 |
| | 1 | 15:00 | 275 | 10 |
| | 2 | 14:30 | 1685 | 12 |
| | 3 | 13:00 | 3035 | 13 |
| 2 | 1 | 10:25 | 0 | 0 |
| | 1 | 11:00 | 35 | 24 |
| | 1 | 15:00 | 275 | 55 |
| | 2 | 14:30 | 1685 | 67 |
| | 3 | 13:00 | 3035 | 67 |

FIG. 4 shows gel strength over time with 2% GENU® pectin CS 30 and different concentrations of calcium citrate. The gelation is completed after about 4 hours.

Table 20 shows the data relating to gel strength over time when using tri-calcium phosphate.

TABLE 20
Gel strength over time with tri-calcium phosphate.

| Sample | Day | Time | Time, min | GS |
|---|---|---|---|---|
| 1 | 1 | 09:35 | 0 | 0 |
| | 1 | 10:05 | 30 | 4 |
| | 1 | 14:50 | 315 | 5 |
| | 2 | 13:35 | 1680 | 5 |
| | 3 | 13:35 | 3120 | 6 |
| 2 | 1 | 09:35 | 0 | 0 |
| | 1 | 10:05 | 30 | 22 |
| | 1 | 14:50 | 315 | 29 |
| | 2 | 13:35 | 1680 | 33 |
| | 3 | 13:35 | 3120 | 34 |
| 3 | 1 | 09:35 | 0 | 0 |
| | 1 | 10:05 | 30 | 55 |
| | 1 | 14:50 | 315 | 86 |
| | 2 | 13:35 | 1680 | 97 |
| | 3 | 13:35 | 3120 | 101 |

FIG. 5 shows the gel strength over time with 2% GENU® pectin CS 30 and different concentrations of tri-calcium phosphate. Again, it takes about 4 hours for the gel to be completed.

Acceptable gel strengths are achieved with 2% GENU® pectin CS 30 and 0.15 g calcium sulphate or 0.20 g calcium hydrogen phosphate or 0.20 g calcium citrate or 0.15 g tri-calcium phosphate.

Tables 12 and 13 indicate that when an oil such as grape fruit oil is used in the gel, the addition of small amounts of a sugar beet pectin solution would be advantageous, particularly when a lower shear mixing device is used. However, when using a fragrance such as Zesty, perfect emulsions can be achieved without the use of sugar beet pectin, even when a lower shear rate mixing device such as a magnetic stirrer is used.

Additionally, it was observed that the gel compositions evaporate completely over time.

Table 21 shows the gelling time in minutes for pectin-calcium salt gel solutions containing sodium hexametaphosphate in amounts of 0, 0.003, 0.005, and 0.007 weight percent of the gel solution.

TABLE 21
Gelling time for Gel Solutions Containing Varying
Amounts of hexametaphosphate.

% Hexametaphosphate

| | 0 | 0.003 | 0.005 | 0.007 |
|---|---|---|---|---|
| Minutes to gel | 2.5 | 45 | 60 | 80 |

FIG. 6 shows the gelling time in minutes for pectin-calcium salt gel solutions containing sodium hexametaphosphate in amounts of 0, 0.003, 0.005, and 0.007 weight percent of the gel solution. As shown in the graph, addition of the sodium hexametaphosphate resulted in a substantially linear increase in gelling time.

Table 22 shows the gelling time in minutes for pectin-calcium salt gel solutions having a pH of about 4, 4.5, 4.75, and 4.83.

TABLE 22
Gelling time for Gel Compositions Having Varying pH.

pH

| | 4 | 4.5 | 4.75 | 4.83 |
|---|---|---|---|---|
| Minutes to gel | 2.2 | 4.76 | 38.5 | 768 |

FIG. 7 shows the gelling time in minutes for pectin-calcium salt gel solutions having a pH of about 4 and above. As shown in the graph, an increase in pH of the gel solution resulted in an increase in gelling time. However, at a pH of about 4.7 or above, this particular gel solution would not gel.

Table 23 shows the gelling time in minutes for pectin gel solutions at a pH of 3.8, 4.04, and 4.4 before adding the tri-calcium phosphate.

TABLE 23
Gelling time for Gel Compositions Containing
Hexametaphosphate Having Varying pH.

| | pH before adding | | |
|---|---|---|---|
| | tri calcium phosphate | | |
| | 3.8 | 4.04 | 4.4 |
| Minutes to gel | 55 | 60.2 | 300 |

FIG. 8 shows the gelling time in minutes for pectin-calcium salt gel solutions containing 0.005 weight percent sodium hexametaphosphate at a pH of about 3.8, 4.04, and 4.4. As shown in the graph, use of a sequestrant in combination with a pH adjustor resulted in an additional increase in gelling time, over the sequestrant or pH adjustor alone.

FIG. 9 shows the gel strength of gels containing 0.0025 weight percent sodium hexametaphosphate that have been stirred for different time periods. These results show that these particular gel solutions may be stirred for up to about 30 minutes.

Publications cited herein and the materials for which they are cited are specifically incorporated by reference herein. Modifications and variations of the methods and compositions described herein will be obvious to those skilled in the art from the foregoing detailed description. Such modifications and variations are intended to come within the scope of the appended claims.

What is claimed is:

1. An air freshening gel composition, comprising:
a low ester pectin in an amount from about 0.5 to about 2.5 percent by weight of the gel composition;
a substantially insoluble calcium salt in an amount from about 0.1 to about 0.50 percent by weight of the gel composition;
water; and
fragrance.

2. The composition of claim 1, wherein the low ester pectin has a degree of esterification of about 25 to about 40.

3. The composition of claim 1, wherein the substantially insoluble calcium salt comprises calcium citrate, tri-calcium phosphate, calcium sulfate, calcium hydrogen phosphate, or a combination thereof.

4. The composition of claim 1, wherein:
the low ester pectin is present in an amount from about 1.0 to about 2.0 percent by weight of the gel composition, and
the calcium salt is present in an amount from about 0.1 to about 0.4 percent by weight of the gel composition.

5. The composition of claim 1, wherein the water comprises demineralized water.

6. The composition of claim 1, wherein the fragrance is present in an amount from about 4.0 to about 6.0 percent by weight of the gel composition.

7. The composition of claim 1, wherein the fragrance comprises grape fruit oil, lemon oil, or a combination thereof.

8. The composition of claim 1, wherein the gel composition has a gel strength from about 5 grams to about 250 grams.

9. The composition of claim 1, wherein the gel composition has a pH from about 3.0 to about 5.5.

10. A method of making an air freshening gel composition, comprising:
dissolving a low ester pectin in demineralized water to form a pectin solution;
suspending a substantially insoluble calcium salt in demineralized water to form a salt suspension;
combining the pectin solution and the salt suspension to form a gel solution; and
allowing the gel solution to form a gel composition,
wherein the low ester pectin is present in the gel solution in an amount from about 0.5 to about 2.5 percent by weight of the gel solution, and the calcium salt is present in the gel solution an amount from about 0.1 to about 0.50 percent by weight of the gel solution.

11. The method of claim 10, wherein the demineralized water is at room temperature.

12. The method of claim 10, wherein the dissolving and/or suspending steps comprise stirring.

13. The method of claim 10, wherein suspending the calcium salt in demineralized water further comprises dispersing a fragrance in the demineralized water with the calcium salt to form the salt suspension.

14. The method of claim 10, wherein the gel composition is formed from the gel solution in from about 2 to about 5 minutes.

15. The method of claim 10, further comprising adding a sugar beet pectin solution to the pectin solution prior to combining the pectin solution and the salt suspension.

16. The method of claim 15, wherein the sugar beet pectin solution comprises sugar beet pectin in an amount from about 1.0 to about 3.0 percent by weight of the sugar beet pectin solution.

17. The method of claim 10, wherein the low ester pectin has a degree of esterification of about 25 to about 40.

18. The method of claim 10, wherein the substantially insoluble calcium salt comprises calcium citrate, tri-calcium phosphate, calcium sulfate, calcium hydrogen phosphate, or a combination thereof.

19. The method of claim 10, wherein:
the low ester pectin is present in an amount from about 1.0 to about 2.0 percent by weight of the gel solution, and
the calcium salt is present in an amount from about 0.1 to about 0.4 percent by weight of the gel solution.

20. The method of claim 13, wherein the fragrance is present in the gel solution in an amount from about 4.0 to about 6.0 percent by weight of the gel solution.

21. The method of claim 13, wherein the fragrance comprises grape fruit oil, lemon oil, or a combination thereof.

22. The method of claim 10, further comprising adding a calcium-stabilizing sequestrant and/or a pH adjustor to the gel solution to increase a gel formation time of the gel composition.

23. The method of claim 22, wherein adding the calcium-stabilizing sequestrant and/or the pH adjustor to the gel solution comprises adding the calcium-stabilizing sequestrant and/or the pH adjustor to the pectin solution.

24. The method of claim 10, further comprising adding sodium hexametaphosphate to the pectin solution, wherein the sodium hexametaphosphate is present in the gel solution in an amount from about 0.001 to about 0.01 percent by weight of the gel solution.

25. The method of claim 10, further comprising adding sodium hydroxide to the pectin solution, wherein the sodium hydroxide is present in the gel solution in an amount effective to bring a pH of the gel solution within a range from about 4.0 to about 4.7.

26. A method of making an air freshening gel composition, comprising:
dissolving a low ester pectin in water to form a pectin solution;
adding a calcium salt to the pectin solution to form a gel solution; and
allowing the gel solution to form a gel composition,
wherein the low ester pectin is present in the gel solution in an amount from about 0.5 to about 2.5 percent by weight of the gel solution, and the calcium salt is present in the gel solution an amount from about 0.1 to about 0.50 percent by weight of the gel solution.

27. The method of claim 26, further comprising adding a fragrance to the pectin solution to form the gel solution.

28. The method of claim 27, wherein the fragrance is present in the gel solution in an amount from about 4.0 to about 6.0 percent by weight of the gel solution.

29. The method of claim 26, further comprising adding a sugar beet pectin to the pectin solution.

30. The method of claim 26, wherein the water comprises demineralized water.

31. The method of claim 26, further comprising adding a calcium-stabilizing sequestrant and/or a pH adjustor to the gel solution to increase a gel formation time of the gel composition.

32. The method of claim 31, wherein adding the calcium-stabilizing sequestrant and/or the pH adjustor to the gel solution comprises adding the calcium-stabilizing sequestrant and/or the pH adjustor to the pectin solution.

33. The method of claim 26, further comprising adding sodium hexametaphosphate to the pectin solution, wherein the sodium hexametaphosphate is present in the gel solution in an amount from about 0.001 to about 0.01 percent by weight of the gel solution.

34. The method of claim 26, further comprising adding sodium hydroxide to the pectin solution, wherein the sodium hydroxide is present in the gel solution in an amount effective to bring a pH of the gel solution within a range from about 4.0 to about 4.7.
